(19) United States
(12) Patent Application Publication
(10) Pub. No.: US 2020/0078541 A1
(43) Pub. Date: Mar. 12, 2020

(54) AEROSOLIZER DOCKING STATION AND INDIVIDUAL AEROSOLIZATION POD FOR INTERCHANGEABLE USE THEREIN

(71) Applicant: Airganics, LLC., Clearview, FL (US)

(72) Inventors: Frank Lewis, St. Petersburg, FL (US); Joshua Matzkin, Tampa, FL (US); Carol Sullivan, St. Petersburg, FL (US)

(73) Assignee: Airganics, LLC., Clearview, FL (US)

(21) Appl. No.: 16/126,571

(22) Filed: Sep. 10, 2018

(51) Int. Cl. A61M 15/00; A61M 11/00; A61M 16/00

(52) U.S. Cl. CPC A61M 15/0083; A61M 11/005; A61M 15/0086; A61M 16/024; A61M 15/0071; A61M 15/0085

(57) ABSTRACT

An aerosolizer docking station comprising an adjustable docking port configured to receive an individual aerosolizer, at least one air intake port to receive air, an air pump configured to pump the received air to the individual aerosolizer via an air hose, the pumped air facilitating aerosolization of liquid contained in the individual aerosolizer for dispersion to an ambient area, and processing circuitry including a memory and a processor, the memory in communication with the processor, the memory having instructions that, when executed by the processor, configure the processor to control and monitor delivery of the aerosolized liquid from the individual aerosolizer.

### TECHNICAL FIELD

The present disclosure relates to aerosolizer docking stations and more particularly to an aerosolizer docking station having time and duration controls that can accommodate any individual aerosolizer device for in situ use while interchangeably also accommodating liquid-filled aerosolizer cartridges.

### BACKGROUND

Inhalation therapy, in the form of the inhalation of aerosolized liquids, has gained recent popularity. Aromatherapy diffusers and other aerosolization devices can be used for the delivery of essential oils, herbs, vitamins, Tetrahydrocannabinol (“THC”), Cannabidiol (“CBD”), nicotine, medicines and any other substances that can be aerosolized. Individual-use aerosolization devices also provide an effective way to deliver medicaments or other types of aerosolized substances directly to a user.

Typical aerosolization devices include a chamber for housing the liquids that are to be aerosolized and a delivery mechanism whereby a user can inhale the aerosolized liquid. However, direct delivery of the aerosolized liquid in this fashion may not always be desired. Often, in situ aerosolization may be desirous, and it may be preferred to dispense the aerosolized liquids into an ambient space, rather than individually and one at a time through an aerosolization device. For example, a number of people in a room may wish to partake in an aromatherapy session and the use of individual inhalation devices would not be optimal. Individual aerosolization devices cannot be used for this purpose. Currently, there is no adequate mechanism that allows a user to alternate between a personal inhalation device and an aerosolizer docking station.

As a further example of current aerosolization devices, aromatherapy diffusers are either mechanical or ultrasonic. If the diffuser is mechanical, then the end-user must buy essential oils separately and add the essential oils to the pad (wick) inside the diffuser. If the diffuser is ultrasonic, then the end-user must add water and essential oils. This can be cumbersome and inefficient.

What is therefore needed is a docking station that improves upon the deficiencies of prior art aerosolization devices.

### SUMMARY

The apparatus described in the present disclosure allows one to be able to go from a personal inhalation device to a docking station (and vice versa). For example, a person may drive to work using an individual (and easily portable) vaporizer pen (“vape pen”) with energizing essential oils and then dock the device when they get to their office, thus allowing them to continue to use and enjoy the benefits of the vape pen while working (which can be shared or not shared with someone else). The apparatus of the present disclosure makes aerosolization more convenient and also easier. With the apparatus described in the present disclosure, the oil is already inside the inhalation device or the inside the pod, ready for use.

According to one aspect of the disclosure, an aerosolizer docking station is provided. The docking station includes an adjustable docking port configured to receive an individual aerosolizer, at least one air intake port to receive air, an air pump configured to pump the received air to the individual aerosolizer via an air hose, the pumped air facilitating aerosolization of liquid contained in the individual aerosolizer for dispersion to an ambient area, and processing circuitry including a memory and a processor, the memory in communication with the processor, the memory having instructions that, when executed by the processor, configure the processor to control and monitor delivery of the aerosolized liquid from the individual aerosolizer.

According to another aspect of the disclosure, an aerosolizer docking station is provided. The docking station includes a docking port configured to receive an individual aerosolizer, the individual aerosolizer having at least one air intake port for receiving air, an intake conduit configured to receive aerosolized liquid from the individual aerosolizer, an aerosol exit tube configured to disperse aerosolized liquid to an ambient area, an air pump having a first end and a second end, the air pump connected to the intake conduit at the first end and the aerosol exit tube at the second end, the pump configured to pump the aerosolized liquid from the intake conduit through the aerosol exit tube, and processing circuitry including a memory and a processor, the memory in communication with the processor, the memory having instructions that, when executed by the processor, configure the processor to control and monitor delivery of the aerosolized liquid dispersed from the aerosol exit tube.

According to yet another aspect of the disclosure, an aerosolizer docking station is provided where the docking station includes an adjustable docking port configured to receive an individual aerosolizer, a power source, at least one electrode for providing an electrical connection with the individual aerosolizer, the power source providing power to the individual aerosolizer via the electrical connection if the individual aerosolizer does not have its own power source, at least one air intake port to receive air, and an air pump configured to pump the received air to the individual aerosolizer via an air hose, the pumped air facilitating aerosolization of liquid contained in the individual aerosolizer for dispersion to an ambient area. The docking station further includes processing circuitry including a memory and a processor, the memory in communication with the processor, the memory having instructions that, when executed by the processor, configure the processor to determine at least one of a duration of time of an aerosolization, a duration of time between successive aerosolizations, a number of total aerosolizations within a predetermined time period, a time when a next allowed aerosolization is permitted for a particular use, and a time when an aerosolization at least one of began and ended. The docking station also includes a display for displaying at least one of the duration of time of an aerosolization, the duration of time between successive aerosolizations, the number of total aerosolizations within a predetermined time period, the time when the next allowed aerosolization is permitted for a particular user, and the time when an aerosolization at least one of began and ended.

### BRIEF DESCRIPTION OF DRAWINGS

A more complete understanding of the present embodiments, and the attendant advantages and features thereof, will be more readily understood by reference to the following detailed description when considered in conjunction with the accompanying drawings wherein:

FIG. 1 is a front view of the internal components of the aerosolizer docking station in accordance with an embodiment of the present disclosure;

FIG. 2 is a front view of the external components of the aerosolizer docking station in accordance with the embodiment of FIG. 1;

FIG. 3 is a front view of the internal components of the aerosolizer docking station in accordance with an alternate embodiment of the present disclosure;

FIG. 4 is a front view of the external components of the aerosolizer docking station in accordance with the embodiment of FIG. 3; and

FIG. 5 is a front view of the external components of the aerosolizer docking station in accordance with yet another embodiment of the present disclosure.

### DETAILED DESCRIPTION OF EXEMPLARY EMBODIMENTS

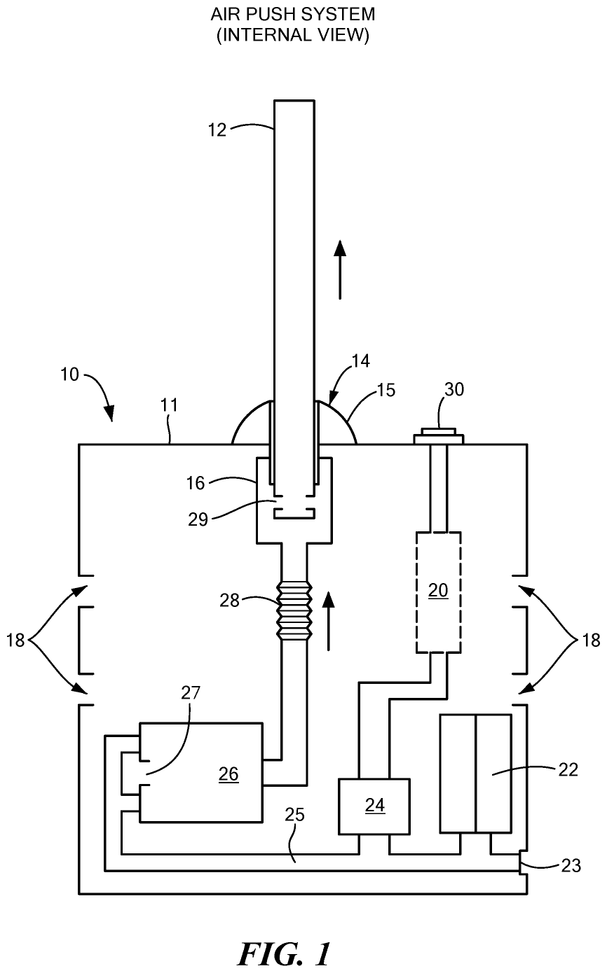

Referring to FIGS. 1-5, the aerosolizer docking station of the present disclosure is designed to house an individual aerosolizer device and dispense aerosolized liquids to a surrounding area. Referring to FIG. 1, in accordance with one embodiment of the present disclosure, an aerosolizer docking station 10 including a housing 11 configured to receive and secure an individual aerosolizer device 12, is shown. Individual aerosolizer device 12 may be of any shape or size and the dimensions and shape of individual aerosolizer device 12 shown in FIG. 1 is meant to be exemplary only. A docking port 14 receives and secures individual aerosolizer device 12 within housing 11. Docking port 14 may be provided in various different configurations in order to receive and secure individual aerosolizer devices 12 of varying dimensions, shapes and types.

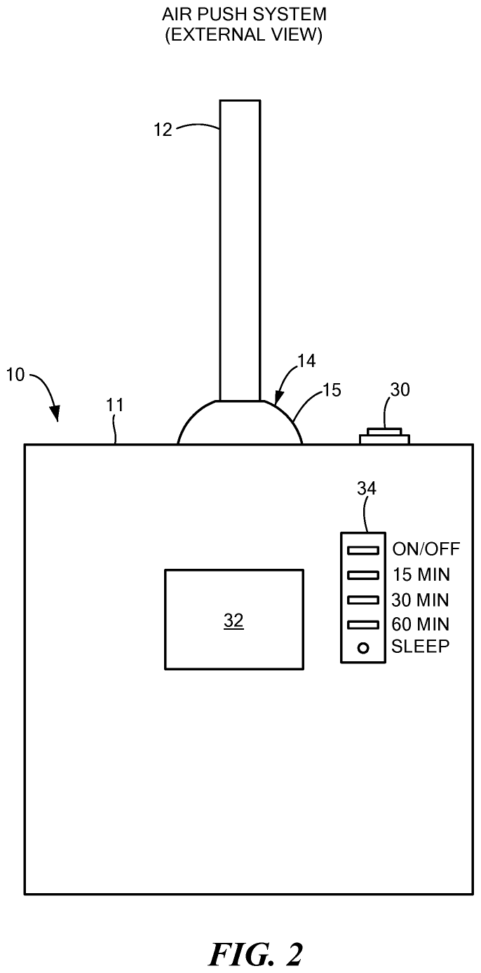

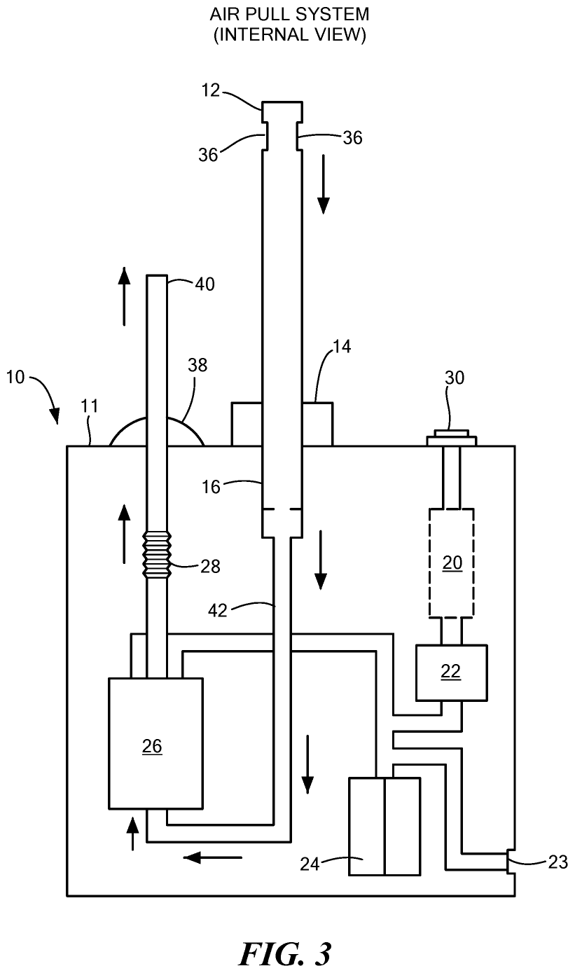

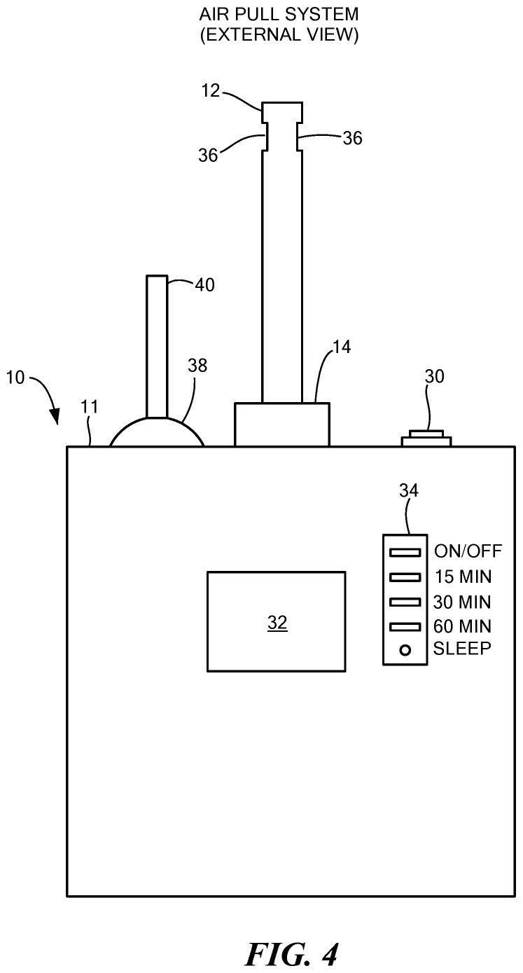

In one embodiment, docking port 14 may include swivel member 15. Once individual aerosolizer device 12 is inserted within docking port 14, swivel member 15 allows individual aerosolizer device 12 to be rotated 360 degrees. Advantageously, this allows for the dispensing of aerosolized liquids in various directions. Individual aerosolizer device 12 may be configured to aerosolize any type of liquid, for example, nutrients, vitamins, herbs, oils, aromatic waters, homeopathics, nutraceuticals and medicines. Docking port 14 may also include docking collar 16, which secures individual aerosolizer device 12 within housing 11. Thus, a user desiring to use dispense aerosolized liquids into a surrounding area, for example, during an aromatherapy session, would insert an individual aerosolizer device 12 within the docking port 14 of the housing 11 of aerosolizer docking station 10. As will be explained below, aerosolized liquid is dispensed to the surrounding area either through individual aerosolizer device 12 (as in the embodiment shown in FIGS. 1 and 2) or through an aerosol exit tube 40 in aerosolizer docking station 10 (as in the embodiment shown in FIGS. 3 and 4).

The embodiment depicted in FIGS. 1 and 2 relates to an air “push” system whereby aerosolizer docking station 10 receives air from the outside surroundings through air intake holes or ports 18. The air enters the interior or housing 11 from air intake ports 18 and then enters a pump 26 through pump intake port 27. Pump could be, for example, a DC motor pump, although the present disclosure is not limited to a specific type of pump and any compatible pump may be used. Pump 26 pumps the air received through pump intake port 27 through hose 28 and then through aerosolizer air intake ports 29 on individual aerosolizer device 12. The air entering individual aerosolizer 12 activates a pressure switch which activates the heat coil and aerosolizes the liquid contained within individual aerosolizer 12. The aerosolized liquid is then dispersed in to the ambient air. Aerosolizer docking station 10 is not limited for use with a particular type of individual aerosolizer 12 nor is it limited to work with individual aerosolizers that aerosolize liquid in the manner explained above. For example, aerosolizer docking station 10 is equally adaptable to work with individual aerosolizers or vaporizers that aerosolize liquid via other means, for example, via ultrasound.

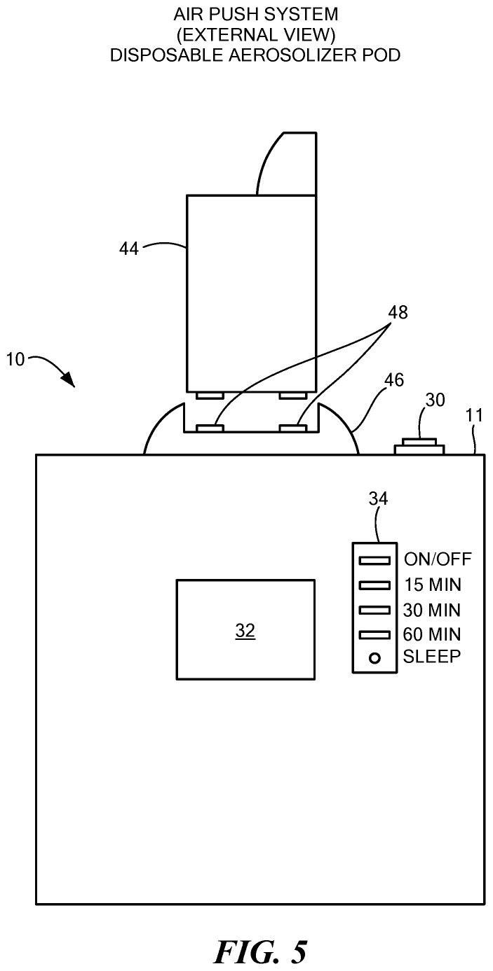

Continuing to refer to FIG. 1, housing 11 of aerosolizer docking station 10 includes battery 22 and microprocessor 24. Battery 22 may provide the power to aerosolizer docking station 10 and in certain instances to individual aerosolizer 12 (as shown in FIG. 5 and discussed below). Battery 22 may be any type of battery including a rechargeable battery, which will allow aerosolizer docking station 10 to be charged, removed from the charger and be transported, thus making the docking station 10 fully portable. Alternately, housing 11 may include a USB connector port 23 and receive power via an external power source via a USB connector. Microprocessor 24 includes processing circuitry and a memory, the memory in communication with microprocessor 24, the memory having instructions that, when executed by the microprocessor 24, configure microprocessor 24 to perform a number of tasks associated with the control and monitoring of delivery of the aerosolized liquid from individual aerosolizer 12. Battery 22, microprocessor 24 and pump 26 are in electrical communication with each other through wires in conduit 25. Thus, microprocessor 24 may be programmed to perform a variety of tasks, discussed in greater detail below, which may include determining a duration of time of an individual aerosolization, a duration of time between successive aerosolizations, a number of total aerosolizations within a predetermined time period, a time when a next allowed aerosolization is permitted for a particular use, and a time when an aerosolization has started or has ended. These settings may be set by the manufacturer prior to aerosolizer docking station 10 being delivered to the end-user, or may be programmed directly by the end-user.

In one embodiment, housing 11 also includes a burst button 30, which provides an instant burse of aerosol from individual aerosolizer 12. Thus, utilizing burst button 30, a user can override the programmed time intervals that the aerosolized liquid is to be dispersed. Activation of burst button 30 provides a burst of air to be sent to motor pump 26 and directed towards individual aerosolize 12 via hose 28. Housing 11 may also include a series of timing switches, the inside 20 of which can be seen in FIG. 1. The timing switches and their functions are discussed in further detail below with respect to FIG. 2.

FIG. 2 is a front view of the external components of aerosolizer docking station 10 in accordance with the embodiment shown in FIG. 1 and discussed above. In one embodiment, housing 11 includes a display 32 and timing switches 34. Timing switches 34 could include an on/off switch which powers up and powers down docking station 10 and switches to set the length of aerosol dispersal duration, the time intervals between successive aerosolizations and the total number of aerosolizations in a given time period. For example, a user may want the liquid in individual aerosolizer 12 to be aerosolized and dispersed for a duration of 10 minutes in intervals of 15 minutes, 30 minutes or 60 minutes. Or the setting can limit the total number of aerosolizations in a one hour period to, for example, five. The intervals set by the switches shown in FIG. 2 are exemplary only, and thus the dispersion time intervals can be set to any time limit. Timing switches can also include a sleep mode. In this fashion, the aerosolizer docking station 10 can be programmed to allow the inserted individual aerosolizer 12 to disperse aerosolized liquid at various time intervals and for various durations while the user sleeps. The exterior of the housing 11 may also include a low battery indicator to indicate that battery 22 needs to be replaced.

In one embodiment, display 32 may be disposed on the exterior of housing 11 of aerosolizer docking station 10. Display 32 may display various settings that have been selected by the user (or pre-programmed) via timing switches 34 thus allowing a user to quickly and easily see what the current settings are. These may include, for example, the time duration of each aerosol dispersion, the time between aerosol dispersions, the total number of aerosol dispersions within a given time period, and the time until the next allowed aerosol dispersion. Different settings can be selected for different users. Display 32 may be, in one embodiment, an LED display. In other embodiments, the various displays (e.g., time duration of each aerosol dispersion, the time between aerosol dispersions, the total number of aerosol dispersions within a given time period, and the time until the next allowed aerosol dispersion) may be represented in a different, unique color. The present disclosure is not limited to what may be displayed on display 32 and other settings, other than those discussed herein, may be displayed.

In yet another embodiment, aerosolizer docking station 10 may be controlled remotely via a remote control device or a mobile device such as a cellular/smart phone. For example, a person can activate/deactivate docking station 10, set or change the settings on the timing switches including, for example, setting or changing the aerosol dispersion time intervals and activating or deactivating sleep mode, all via a wireless connection with the user's remote control device or mobile phone.

What follows is a non-limiting exemplary use case, describing how aerosolizer docking station 10 can be programmed via timing switches 34 to allow multiple users to use aerosolizer docking station 10 with different individual aerosolizers 12. For example, during a first aromatherapy session, User A may be using individual aerosolizer 12 with certain medicaments, herbs, or nutrients and requires these medicaments to be dispersed and inhaled at selected time intervals and for selected durations. User A can have the settings entered by using timing switches 34 to program aerosolizer docking station 10 to provide aerosolized liquid to be dispersed from individual aerosolizer 12 at prescribed times. Thus, in one non-limiting scenario, timing switches 34 are used to program aerosolizer docking station 10 program for individual aerosolizer 12 to provide aerosol bursts of 15 minute durations, with 15 minute intervals between successive aerosol bursts. For example, this might occur twice a day, in the morning and then again in the early evening. Thus, User A may inhale the aerosol starting at 7:30 AM until 8:45 AM at the selected durations and intervals. This cycle may then be programmed to be repeated in the evening between 6 PM and 7 PM.

User B, perhaps someone living in the same house as User A, may be using a different individual aerosolizer 12 for their own aromatherapy session. For example, User B enjoys aromatherapy in the evenings after a long day of work. User B can remove the first individual aerosolizer 12 that was being used by User A and replace it with a second individual aerosolizer 12 that contains essential oils that, once aerosolized, provide pleasing aromas to the surrounding area. Advantageously, aerosolizer docking station 10 can accommodate the second individual aerosolizer 12 by either an adjustment of docking port 14 to receive the second individual aerosolizer 12 which may have a different configuration or be of a different size than first individual aerosolizer 12, or by replacing docking port 14 with a different docking port 14 to accommodate individual aerosolizers of different dimensions and sizes. Aerosolizer docking station 10 may be programmed specifically for User B to allow the second individual aerosolizer 12 to release aerosolized liquid for an aromatherapy session starting at, for example, 8:30 PM, for a duration of 20 minute, with 15 minute intervals in between, ending at 10 PM, when aerosolizer docking station 10 enters sleep mode, and aerosolizer docking station 10 shuts off. The sessions for User A and User B may be repeated again the next evening or for any period of time.

Referring to FIG. 3, an alternate embodiment of aerosolizer docking station 10 of the present disclosure is shown. The embodiment shown in FIG. 3 is an air “pull” embodiment. In this embodiment, pump 28 is reversed and instead of pushing compressed air up through and out of individual aerosolizer 12 so individual aerosolizer 12 can disperse the aerosolized liquid to the ambient air, air enters individual aerosolizer 12 and the aerosolized liquid contained therein is “pulled” by pump 28 through individual aerosolizer 12 and the aerosolized liquid drawn through pump 28 is dispersed through aerosol exit tube 40.

In the embodiment depicted in FIG. 3, air is received through aerosolizer intake ports 36 located on individual aerosolizer 12. The air then activates the aerosolization process in individual aerosolizer 12 where the liquid stored therein is aerosolized and then pulled down into aerosolizer docking station 10 by pump 26 through aerosolized liquid intake conduit 42. Thus, pump 26 is configured to act in the reverse manner in which it did in the embodiment depicted in FIGS. 1 and 2. Pump 26, having pulled the aerosolized mixture within aerosolizer docking station 10 then forces the aerosolized mixture though air hose 28 where it is expelled through aerosol exit tube 40 and into the ambient air. Thus, this embodiment differs from the “push” system illustrated in FIGS. 1 and 2 whereby in this embodiment, air is “pulled” through aerosolizer docking station 10 and directed up and out through a different exit means, i.e., aerosol exit tube 40. As in the earlier embodiment, aerosolizer docking station 10 may include a swivel 38 at the base of aerosol exit tube 40 to allow for exit tube 40 to be maneuvered at various angles, thus dispersing the aerosol to virtually any direction within the surrounding area.

As in the earlier embodiment, contained within housing 11 are a battery 22 and/or USB connector port 23 which allows docking station 10 can be power via a remote power source. Docking port 14 receives individual aerosolizer 12 and may be adjusted to receive individual aerosolizers of different sizes and dimensions. Alternately, docking port 14 may be adjusted to account for differently sized individual aerosolizers. Docking collar helps secure individual aerosolizer 12 within docking station 10. Swivel 38 allows aerosol exit tube 40 to be rotated in various directions such that aerosol existing aerosol exit tub 40 can be directed in any direction the user wishes. A series of timing switches (the inside frame of which is seen in FIG. 3 and as 20), in conjunction with microprocessor 24 can be used in the same fashion as described above and illustrated in FIGS. 1 and 2. Further, burst button may be used to provide an additional burst of air that pump 26 utilizes to force additional air through hose 28 to facilitate the pumping of aerosolized liquid up through hose 28 and out of aerosol exit tube 40.

FIG. 4 depicts the external components of the air “pull” system described above and illustrated in FIG. 3. As in the earlier embodiment, housing 11 may include a timer panel 34 that includes a series of timing switches that can be set by the user, or pre-set in a manner described above. In one embodiment, a display 32 may allow the various timing settings to be displayed. As in the air “push” embodiment, display 32 may be an LED display and may include a color coding scheme to identify different parameters, i.e., duration of an individual dosage, time interval between dosages, total number of dosages within a predetermined amount of time, time before next allowable dosage, etc.

An alternate embodiment of the present disclosure is illustrated in FIG. 5. In certain instances, the individual aerosolizer may be in the form of an aerosolizer cartridge or “pod” 44, which does not have a power source and thus cannot aerosolize the liquid contained therein without external power. In this embodiment, aerosolizer docking station 10 can act as a module that supplies the power to the aerosolizer pod. In this arrangement, aerosolizer docking station 10 can include a pod-receiving docking port 46 which is specially configured to receive an aerosolizer pod 44, as shown in FIG. 5. Aerosolizer pod 44 is configured to house any product that can be aerosolized such as, but not limited to, essential oils, herbs, vitamins, THC, CBD, nicotine, and medicines. In this embodiment, pod-receiving docking port 46 of aerosolizer docking station 10 includes one or more electrical contacts 48, i.e., electrodes, in order to provide an electrical connection between aerosolizer docking station 10 and aerosolizer pod 44. Power can then be supplied to aerosolizer pod 44 from battery 22 or from a remote power supply through, for example, USB connector port 23 (shown in FIGS. 1 and 3) through the electrical connection established by electrical contacts 48.

Thus, the embodiment illustrated in FIG. 5 and described above advantageously provides a user with the ability to pair aerosolizer docking station 10 with a variety of different types of individual aerosolizers 12, including those aerosolizers that do not have their own power source, i.e., aerosolizer pod 44. Because some aerosolizers may not include their own power supply, housing 11 includes electrical contacts 48, which provide the electrical connection with the aerosolizer pod 44 and battery 22 or an external power source may be used to supply the power to aerosolizer pod 44, which will then enable the liquid contained within aerosolizer pod 44 to be aerosolized and released into the ambient air. The embodiment depicted in FIG. 5 may be applicable to both the air intake “push” system shown in FIGS. 1 and 2 as well as the air intake “pull” system shown in FIGS. 3 and 4.

Aerosolizer docking station 10 is advantageously configured to receive and secure an individual aerosolizer 12 as shown in FIGS. 1-4 as well as an aerosolizer pod 44, as shown in FIG. 5. In one embodiment, if the user wishes to host an individual aerosolizer 12, docking port 14 is secured to housing 11. However, if the user instead wishes to host an aerosolizer pod 44 instead, docking port 14 can be removed and replaced by pod-receiving docking port 46, which is configured to receive aerosolized pod 44. Thus, pods 44 may be used separately in a modular aerosolization device or be inserted within docking station 10 for in situ dispersion of the aerosolized liquid therein.

In one embodiment, aerosolizer pods 44 are designed to only be compatible with aerosolizer docking station 10. In one embodiment, docking station 10 can be packaged with an empty aerosolizer pod 44, and the user can separately purchase different liquids to be aerosolized. In another embodiment, aerosolizer pod 44 can be pre-filled with any type of aromatherapy liquid, much as coffee refill cups can be filled with any type of coffee. In another embodiment, aerosolizer pod 44 is purchased separately, and used with aerosolizer docking station 10. The present disclosure does not limit aerosolizer pod 44 to a particular shape, size, or configuration. Pod 44 includes a housing sized to receive liquid for aerosolization, and to be received and secured by pod-receiving docking port 46 of aerosolizer docking station 10. Battery 22 or an external power supply supplies the electrical power to pod 44, and electrical contacts 48 provide the electrical connection between aerosolizer docking station 10 and aerosolizer pod 44.

Pods 44 may aerosolize the liquid contained therein via the use of heating coils or by ultrasound. The use of heating coils or ultrasound to aerosolize the liquid does not affect how pods 44 are affixed to docking station 10. In either embodiment, pods 44 are attached to docking station 10 via pod-receiving docking port 46 and electrical contacts 48 and receive power from battery 22 (or an external power source) of docking station 10. If pod 44 is to aerosolize the liquid using ultrasound, pod 44 includes an ultrasonic nebulizer module electrically connected to battery 22 in aerosolizer docking station 10. As is known in the art, ultrasonic nebulizer module includes a vibration module, where the ultrasonic nebulizer module uses ultrasonic frequency to vibrate the vibration module to release particles of the liquid.

Using an ultrasonic pod 44 produces smaller aerosolized droplets than a heating coil pod 44, and will also eliminate the use of propylene glycol that is used with the heating coil pod 44. Ultrasonic pods 44 may be used interchangeably with an ultrasonic dosage vaporizers.

In a non-limiting use-based example, a user may use docking station 10 in the following manner. For example, a user, while driving to work in the morning, may initiate an aromatherapy session by using an individualized individual aerosolizer 12 to diffuse a preferred aerosolized oil. When the user gets to their office, they may wish to continue their aromatherapy session using the same aerosolized oils but instead of using an individual aerosolizer 12, may wish to have the oils dispersed within their office. The user inserts the individual aerosolizer 12 within docking station 10 in the manner described above. The oils are aerosolized in situ such that the user can enjoy the session while working and/or for others to enjoy when they visit the user in the office. On the ride home that evening, the user can simply remove the individual aerosolizer 12 from docking station 10 and continue the session on the drive home using only the individual aerosolizer 12.

In another non-limiting use-based example, a user purchases an individual pod 44 for use with docking station 10. The pod 44 is designed to be compatible only with docking station 10. Pod 44 contains a particular liquid that the user wishes to aerosolize for an aromatherapy session. The user can use pod 44 in an individual aerosolizer 12 and/or also use pod 44 with docking station 10 in the manner described above, thus creating a “mod-pod” configuration where pods 44 can be used interchangeably with an individual aerosolizer 12 or docking station 10 of the present disclosure. The user can purchase different pods 44, each containing a different liquid, to thus create a variety of different aromatherapy sessions.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

It will be appreciated by persons skilled in the art that the embodiments described herein are not limited to what has been particularly shown and described herein above. In addition, unless mention was made above to the contrary, it should be noted that all of the accompanying drawings are not to scale. A variety of modifications and variations are possible in light of the above teachings.

1. An aerosolizer docking station comprising:
an adjustable docking port configured to receive an individual aerosolizer;
at least one air intake port to receive air;
an air pump configured to pump the received air to the individual aerosolizer via an air hose, the pumped air facilitating aerosolization of liquid contained in the individual aerosolizer for dispersion to an ambient area; and
processing circuitry including a memory and a processor, the memory in communication with the processor, the memory having instructions that, when executed by the processor, configure the processor to control and monitor delivery of the aerosolized liquid from the individual aerosolizer.

2. The aerosolizer docking station of claim 1, wherein the processor is further configured to determine at least one of a duration of time of an aerosolization, a duration of time between successive aerosolizations, a number of total aerosolizations within a predetermined time period, a time when a next allowed aerosolization is permitted for a particular use, and a time when an aerosolization at least one of began and ended.

3. The aerosolizer docking station of claim 2, further comprising a display for displaying at least one of the duration of time of an aerosolization, the duration of time between successive aerosolizations, the number of total aerosolizations within a predetermined time period, the time when the next allowed aerosolization is permitted for a particular user, and the time when an aerosolization at least one of began and ended.

4. The aerosolizer docking station of claim 1, further comprising a pod-receiving docking port configured to receive and secure a pod containing liquid to be aerosolized, the pod-receiving docking port interchangeable with the adjustable docking port to allow the docking station to accommodate either the individual aerosolizer or the pod.

5. The aerosolizer docking station of claim 4, further comprising:
a power source; and
at least one electrode for providing an electrical connection with the pod, the power source providing power to the pod via the electrical connection.

6. The aerosolizer docking station of claim 4, wherein the pod aerosolizes the liquid contained therein via a heating coil.

7. The aerosolizer docking station of claim 4, wherein the pod aerosolizes the liquid contained therein via ultrasound.

8. The aerosolizer docking station of claim 1, wherein the aerosolized liquid is one of nutrients, vitamins, herbs, oils, aromatic waters, homeopathics, nutraceuticals, and medicines.

9. The aerosolizer docking station of claim 1, further comprising:
a power source; and
at least one electrode for providing an electrical connection with the individual aerosolizer, the power source providing power to the individual aerosolizer via the electrical connection.

10. An aerosolizer docking station comprising:
a docking port configured to receive an individual aerosolizer, the individual aerosolizer having at least one air intake port for receiving air;
an intake conduit configured to receive aerosolized liquid from the individual aerosolizer;
an aerosol exit tube configured to disperse aerosolized liquid to an ambient area;
an air pump having a first end and a second end, the air pump connected to the intake conduit at the first end and the aerosol exit tube at the second end, the pump configured to pump the aerosolized liquid from the intake conduit through the aerosol exit tube; and
processing circuitry including a memory and a processor, the memory in communication with the processor, the memory having instructions that, when executed by the processor, configure the processor to control and monitor delivery of the aerosolized liquid dispersed from the aerosol exit tube.

11. The aerosolizer docking station of claim 10, wherein the processor is further configured to determine at least one of a duration of time of an aerosolization, a duration of time between successive aerosolizations, a number of total aerosolizations within a predetermined time period, a time when a next allowed aerosolization is permitted for a particular use, and a time when an aerosolization at least one of began and ended.

12. The aerosolizer docking station of claim 11, further comprising a display for displaying at least one of the duration of time of an aerosolization, the duration of time between successive aerosolizations, the number of total aerosolizations within a predetermined time period, the time when the next allowed aerosolization is permitted for a particular user, and the time when an aerosolization at least one of began and ended.

13. The aerosolizer docking station of claim 10, further comprising a pod-receiving docking port configured to receive and secure a pod containing liquid to be aerosolized, the pod-receiving docking port interchangeable with the adjustable docking port to allow the docking station to accommodate either the individual aerosolizer or the pod.

14. The aerosolizer docking station of claim 13, further comprising:
a power source; and
at least one electrode for providing an electrical connection with the pod, the power source providing power to the pod via the electrical connection.

15. The aerosolizer docking station of claim 13, wherein the pod aerosolizes the liquid contained therein via a heating coil.

16. The aerosolizer docking station of claim 13, wherein the pod aerosolizes the liquid contained therein via ultrasound.

17. The aerosolizer docking station of claim 10, wherein the aerosolized liquid is one of nutrients, vitamins, herbs, oils, aromatic waters, homeopathics, nutraceuticals, and medicines.

18. The aerosolizer docking station of claim 10, further comprising:
a power source; and
at least one electrode for providing an electrical connection with the individual aerosolizer, the power source providing power to the individual aerosolizer via the electrical connection.

19. A cartridge for use in an aerosolizer docking station, the cartridge comprising:
a housing configured to contain an aerosolizable liquid therein, the housing further configured to be received and secured by a cartridge-receiving docking port of the aerosolizer docking station, the cartridge-receiving docking port interchangeable with an individual aerosolizer docking port to allow the docking station to accommodate either the cartridge or the individual aerosolizer.

20. The cartridge of claim 19, further comprising a heating coil for aerosolizing the liquid contained within the housing, the heating coil powered by a power supply within the aerosolizer docking station.

21. The cartridge of claim 19, further comprising an ultrasonic nebulizer module electrically connected to a power supply in the aerosolizer docking station, the ultrasonic nebulizer module including a vibration module, the ultrasonic nebulizer module using ultrasonic frequency to vibrate the vibration module to release particles of the liquid.

22. A cartridge for use in individual inhalation device, the cartridge comprising:
a housing configured to contain an aerosolizable liquid therein, the housing further configured to be received and secured by a cartridge-receiving docking port of the individual inhalation device.

23. The cartridge of claim 22, further comprising a heating coil for aerosolizing the liquid contained within the housing, the heating coil powered by a power supply within the individual inhalation device.

24. The cartridge of claim 22, further comprising an ultrasonic nebulizer module electrically connected to a power supply in the individual inhalation device, the ultrasonic nebulizer module including a vibration module, the ultrasonic nebulizer module using ultrasonic frequency to vibrate the vibration module to release particles of the liquid.

20. The cartridge of claim 19, further comprising a heating coil for aerosolizing the liquid contained within the housing, the heating coil powered by a power supply within the aerosolizer docking station.

21. The cartridge of claim 19, further comprising an ultrasonic nebulizer module electrically connected to a power supply in the aerosolizer docking station, the ultrasonic nebulizer module including a vibration module, the ultrasonic nebulizer module using ultrasonic frequency to vibrate the vibration module to release particles of the liquid.

22-24. (canceled)
